(19) United States
(12) Patent Application Publication
(10) Pub. No.: US 2021/0100624 A1
(43) Pub. Date: Apr. 8, 2021

(54) IMAGE GUIDED SPINAL SURGERY SYSTEM AND METHOD

(71) Applicant: Warsaw Orthopedic, Inc., Warsaw, IN (US)

(72) Inventors: Erica M. Martelly, Pittsburgh, PA (US); Matthew T. Marrapode, Boulder, CO (US); Jerald L. Redmond, Germantown, TN (US); Tyler R. Koski, Chicago, IL (US)

(21) Appl. No.: 16/592,377

(22) Filed: Oct. 3, 2019

(51) Int. Cl. A61B 34/20; A61B 17/16

(52) U.S. Cl. CPC A61B 34/20; A61B 2034/2065; A61B 17/1635; A61B 17/1671

(57) ABSTRACT

A surgical instrument comprises a tubular member including a tissue engaging element. A shaft is disposable with and movable relative to the tubular member. The shaft includes a tissue gathering element. An image guide is connected with the tubular member and oriented relative to a sensor for registration of anatomical image data and positional tracking of the anatomy during the procedure. In some embodiments, systems, spinal constructs, implants and methods are disclosed.

### TECHNICAL FIELD

The present disclosure generally relates to medical devices for the treatment of musculoskeletal disorders, and more particularly to a surgical system and method for treating a spine.

### BACKGROUND

Spinal disorders such as degenerative disc disease, disc herniation, osteoporosis, spondylolisthesis, stenosis, scoliosis and other curvature abnormalities, kyphosis, tumor, and fracture may result from factors including trauma, disease and degenerative conditions caused by injury and aging. Spinal disorders typically result in symptoms including pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes correction, fusion, fixation, discectomy, laminectomy and implantable prosthetics. As part of these surgical treatments, spinal constructs can be employed with or without interbody devices. Such spinal constructs may include implants such as bone fasteners and vertebral rods to provide stability to a treated region. In some cases, image guided medical and surgical navigation systems provide imaging to assist in surgical procedures and display relative positioning of various body parts and surgical instruments. Surgical instruments are employed, for example, to prepare tissue surfaces and engage implants for disposal with vertebral members. This disclosure describes an improvement over these prior technologies.

### SUMMARY

In one embodiment, a surgical instrument is provided. The surgical instrument comprises a tubular member including a tissue engaging element. A shaft is disposable with and movable relative to the tubular member. The shaft includes a tissue gathering element. An image guide is connected with the tubular member and oriented relative to a sensor for registration of anatomical image data and positional tracking of the anatomy during the procedure. In some embodiments, systems, spinal constructs, implants and methods are disclosed.

In one embodiment, the surgical instrument comprises an outer member including a pin configured to penetrate a selected tissue site of an anatomy. A removable inner member is translatable relative to the outer member and includes a tissue gathering element aligned with the selected tissue site. An image guide is connected with the outer member and oriented relative to a sensor to communicate a signal representative of a position of the outer member relative to the anatomy.

In one embodiment, a surgical system is provided. The surgical system comprises a surgical instrument including an outer member having a tissue engaging element configured to penetrate a selected tissue site of an anatomy and an inner member being relatively translatable relative to the outer member. The inner member includes a tissue gathering element aligned with the selected tissue site. An image guide is connected with the outer member and oriented to communicate a signal representative of a position of the tubular member relative to the anatomy in a navigation field. A tracking device includes a sensor that receives the signal and communicates with a processor to generate data for display of an image from a monitor.

### BRIEF DESCRIPTION OF THE DRAWINGS

The present disclosure will become more readily apparent from the specific description accompanied by the following drawings, in which:

FIG. 1 is a perspective view of components of one embodiment of a surgical system in accordance with the principles of the present disclosure;

FIG. 2 is a perspective view of components of the surgical system shown in FIG. 1;

FIG. 3 is a side view of components of one embodiment of a surgical system in accordance with the principles of the present disclosure;

FIG. 4 is a side view of components of one embodiment of a surgical system in accordance with the principles of the present disclosure;

FIG. 5 is a plan view of components of one embodiment of a surgical system in accordance with the principles of the present disclosure;

FIG. 6 is a plan view of components of one embodiment of a surgical system in accordance with the principles of the present disclosure disposed with an anatomy;

FIG. 7 is a plan view of components of one embodiment of a surgical system in accordance with the principles of the present disclosure disposed with an anatomy;

FIG. 8 is a plan view of components of one embodiment of a surgical system in accordance with the principles of the present disclosure disposed with an anatomy; and

FIG. 9 is a perspective view of components of one embodiment of a surgical system in accordance with the principles of the present disclosure.

### DETAILED DESCRIPTION

The exemplary embodiments of a surgical system are discussed in terms of medical devices for the treatment of musculoskeletal disorders and more particularly, in terms of a surgical system and a method for treating a spine. In some embodiments, the present surgical system comprises an image guided, robot assisted spinal implant system. In some embodiments, the systems and methods of the present disclosure comprise surgical robotic guidance, surgical navigation and medical devices including surgical instruments and implants that are employed with a surgical treatment, as described herein, for example, with a cervical, thoracic, lumbar and/or sacral region of a spine.

In some embodiments, the present surgical system comprises a surgical instrument, for example, a cannulated patient reference for image guidance and bone graft harvesting. In some embodiments, the surgical instrument provides for image-guided patient registration and dynamic tracking. In some embodiments, the surgical instrument provides for harvesting bone during a surgical procedure at a registration site. In some embodiments, a patient reference frame is attached to a patient for registering image data to a patient position and allowing for localization of instruments used in a surgical procedure. In some embodiments, a pin is anchored in the iliac crest, where a frame is mounted onto an anchor for stability. In some embodiments, the iliac crest location can be employed as a target and/or selected location for bone to be harvested for autograft during a surgical procedure. In some embodiments, the surgical instrument includes a patient reference frame having a cannulated pin anchor and a cannulated frame attachment that allows for engagement with a bone harvesting device for collecting autograft during a surgical procedure from a target and/or selected anatomical location.

In some embodiments, the present surgical system comprises a surgical instrument, for example, which includes a cannulated percutaneous pin having a large diameter, a reference frame including an attachment that is cannulated and a bone harvesting cannula that passes through the pin and the frame. In some embodiments, this configuration allows the surgeon to selectively harvest bone at any time during a surgical procedure. In some embodiments, this configuration of the surgical instrument facilitates harvesting bone and placing the percutaneous pin with only one incision.

In some embodiments, the present surgical system comprises a surgical instrument, which is employed with a surgical method that includes the step of placing a percutaneous pin in a selected location and collecting graft from the same selected location. In some embodiments, this configuration of the surgical instrument minimizes injury to the patient. In some embodiments, the surgical instrument includes a patient reference frame attached to an outer tube/cannula, which affixes to the patient's sacra-iliac joint and an inner tube, such as, for example, a bone harvester. In some embodiments, the surgical instrument includes a two-part hollow shaft. In some embodiments, the surgical instrument includes an outer shaft configured as a percutaneous pin for engagement with tissue. In some embodiments, the surgical instrument includes an inner shaft configured for translation and removal from the outer shaft to tamp out harvested tissue. In some embodiments, the surgical instrument includes a navigation component attached with the outer tube and includes a channel that is aligned with the hollow shaft.

In some embodiments, the present surgical system comprises a surgical instrument having a bone harvest element, which is configured as a single patient use instrument that recovers patient bone with minimal tissue disruption and provides a surgeon autologous bone that contains growth factors for bone regeneration. In some embodiments, the surgical instrument harvests tissue from an iliac crest, a proximal femur and/or a distal femur by utilizing a trocar entering through a single incision. In some embodiments, the surgical instrument can be manipulated to re-position the trocar angle to harvest from multiple internal pathways. In some embodiments, the trocar is designed to reduce operative time and blood loss.

In some embodiments, the present surgical system comprises a surgical instrument that is employed with a method of performing robotically-assisted spinal surgery. In some embodiments, the method includes the step of delivering posterior spinal instrumentation through robotic-assisted trajectory alignment tools. In some embodiments, the present surgical system and method includes surgical robotic guidance having robotic software that performs registration of a patient anatomy to a three dimensional working space of a robot.

In some embodiments, the system of the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. In some embodiments, the system of the present disclosure may be employed with other osteal and bone related applications, including those associated with diagnostics and therapeutics. In some embodiments, the disclosed system may be alternatively employed in a surgical treatment with a patient in a prone or supine position, and/or employ various surgical approaches to the spine, including anterior, posterior, posterior mid-line, direct lateral, postero-lateral, and/or antero-lateral approaches, and in other body regions. The system of the present disclosure may also be alternatively employed with procedures for treating the lumbar, cervical, thoracic, sacral and pelvic regions of a spinal column. The system of the present disclosure may also be used on animals, bone models and other non-living substrates, such as, for example, in training, testing and demonstration.

The system of the present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. In some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

As used in the specification and including the appended claims, “treating” or “treatment” of a disease or condition refers to performing a procedure that may include administering one or more drugs to a patient (human, normal or otherwise or other mammal), employing implantable devices, and/or employing instruments that treat the disease, such as, for example, microdiscectomy instruments used to remove portions bulging or herniated discs and/or bone spurs, in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

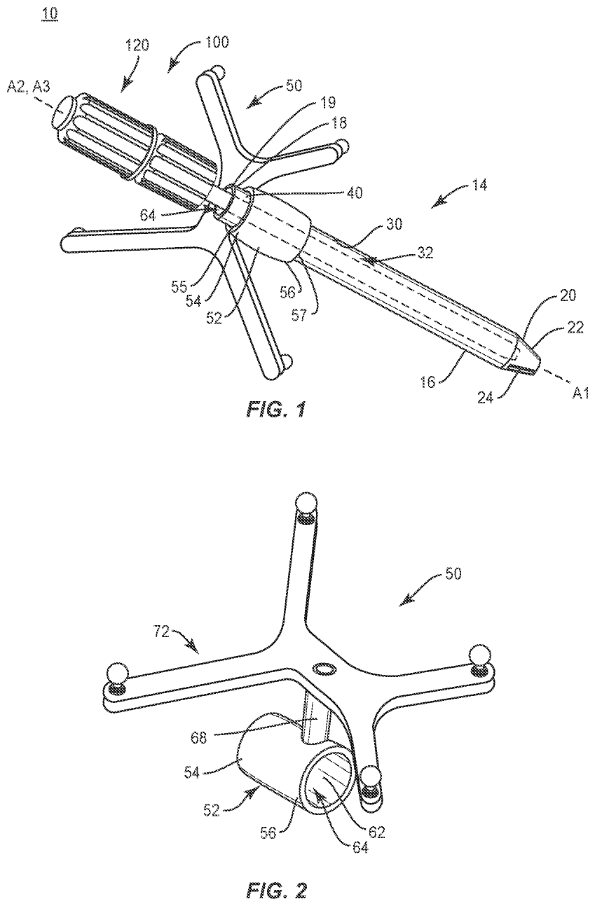

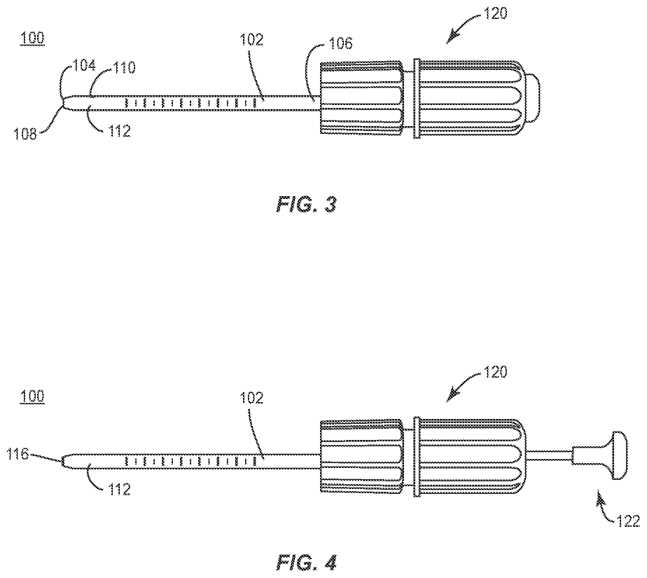

The following discussion includes a description of a surgical system including surgical robotic guidance, surgical navigation, surgical instruments, spinal constructs, implants, related components and methods of employing the surgical system in accordance with the principles of the present disclosure. Alternate embodiments are also disclosed. Reference is made in detail to the exemplary embodiments of the present disclosure, which are illustrated in the accompanying figures. Turning to FIGS. 1-4, there are illustrated components of a surgical system 10.

The components of surgical system 10 can be fabricated from biologically acceptable materials suitable for medical applications, including metals, synthetic polymers, ceramics and bone material and/or their composites. For example, the components of surgical system 10, individually or collectively, can be fabricated from materials such as stainless steel alloys, aluminum, commercially pure titanium, titanium alloys, Grade 5 titanium, super-elastic titanium alloys, cobalt-chrome alloys, superelastic metallic alloys (e.g., Nitinol, super elasto-plastic metals, such as GUM METAL®), ceramics and composites thereof such as calcium phosphate (e.g., SKELITE™), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations.

The components of surgical system 10, individually or collectively, may also be fabricated from a heterogeneous material such as a combination of two or more of the above-described materials. The components of surgical system 10 may be monolithically formed, integrally connected or include fastening elements and/or instruments, as described herein.

Surgical system 10 is employed, for example, with a minimally invasive procedure, including percutaneous techniques, mini-open and open surgical techniques to deliver and introduce instrumentation and/or an implant, such as, for example, a bone fastener, at a surgical site within a body of a patient, for example, a section of a spine. Surgical system 10 includes a surgical instrument 12 configured to facilitate harvesting bone graft from tissue at a patient reference site for image guidance. In some embodiments, such image guidance includes registering image data to a patient position and/or allowing for localization of instruments used in a surgical procedure in connection with surgical robotic guidance and/or surgical navigation.

Surgical instrument 12 includes an outer member, such as, for example, a guide 14 and an inner member, such as, for example, a bone harvester 100. Guide 14 includes a tube 16 that extends between an end 18 and an end 20, and defines an axis A1. End 18 includes a planar end surface 19 having a circumferential configuration and being disposed in a plane substantially perpendicular to axis A1. In some embodiments, surface 19 may be disposed at alternate orientations, relative to axis A1, such as, for example, transverse and/or other angular orientations, such as acute or obtuse, co-axial and/or may be offset or staggered.

End 20 includes a surface 22, which includes a tissue engaging element, such as, for example, a pin 24 extending therefrom. Pin 24 is configured to penetrate tissue to anchor guide 14 with tissue. In some embodiments, surface 22 may include a serrated edge to facilitate engagement of end 20 with tissue. In some embodiments, the tissue engaging element may include other configurations of anchoring elements, such as, for example, a V-shaped wedge configuration or a crescent-shaped configuration. In some embodiments, surface 22 may have alternate surface configurations, such as, for example, smooth, even, rough, threaded for connection with surgical instruments, arcuate, undulating, porous, semi-porous, dimpled, polished, friction and/or textured to enhance fixation with tissue. In some embodiments, surface 22 may be irregular, tapered, offset, staggered, uniform and non-uniform. Engagement of surface 22 with tissue can be flush and/or angled. For example, tube 16 is anchored via penetration of tissue by pin 24. A navigation component 50 is connected with tube 16 and includes a dynamic reference frame and location of the anatomy in a navigation field, as described herein.

In some embodiments, tube 16 includes a feature configured to resist and/or prevent rotation of tube 16 when engaged with tissue. For example, end 20 may include penetrating elements, such as, for example, fins, a nail configuration, barbs, expanding elements, raised elements, ribs, and/or spikes configured to resist and/or prevent rotation of tube 16 relative to tissue.

Tube 16 includes an inner surface 30 that defines a cavity, such as, for example, a guide channel 32. Channel 32 has a substantially linear configuration such that ends 18, 20 are substantially aligned forming a cannulated tube 16. This configuration and alignment facilitates delivery and introduction of a surgical instrument, such as, for example, bone harvester 100, as described herein. In some embodiments, tube 16 is configured to guide bone harvester 100 to resist and/or prevent tissue damage, such as, for example, cervical nerve root damage and vertebral artery injury harvesting. In some embodiments, surface 30 may have various surface configurations, such as, for example, smooth, even, rough, threaded for connection with surgical instruments, arcuate, undulating, porous, semi-porous, dimpled, polished, friction and/or textured. In some embodiments, channel 32 is alternatively oriented relative to axis A1, such as, for example, transverse and/or other angular orientations, such as acute or obtuse, co-axial and/or may be offset or staggered, irregular, tapered, uniform and non-uniform.

A portion of end 18 includes an outer surface 40, which is configured to facilitate engagement with a portion, such as, for example, a tubular attachment 52 of navigation component 50, as described herein. Tubular attachment 52 extends between an end 54 and an end 56 and defines an axis A2. End 54 includes a planar end surface 55 having a circumferential configuration and being disposed in a plane substantially perpendicular to axis A2. In some embodiments, surface 55 may be disposed at alternate orientations, relative to axis A2, such as, for example, transverse and/or other angular orientations, such as acute or obtuse, co-axial and/or may be offset or staggered.

End 56 includes a planar end surface 57 having a circumferential configuration and being disposed in a plane substantially perpendicular to axis A2. In some embodiments, surface 57 may be disposed at alternate orientations, relative to axis A2, such as, for example, transverse and/or other angular orientations, such as acute or obtuse, co-axial and/or may be offset or staggered.

Tubular attachment 52 includes an inner surface 62 that defines a cavity 64. Cavity 64 has a substantially linear configuration such that ends 54, 56 are substantially aligned. Cavity 62 is configured for disposal of end 18 such that surface 62 and surface 40 form a friction fit to fix navigation component 50 with tube 16. In some embodiments, surface 40 is connected with navigation component 50 via an integral connection, pressure fit, interlocking engagement, mating engagement, dovetail connection, clips, barbs, tongue in groove, threaded, magnetic, key/keyslot and/or drill chuck. Axis A2 is coaxially aligned with axis A1 when navigation component 50 is assembled with tube 16, as shown in FIG. 1. This configuration and alignment facilitates attachment with tube 16 delivery and introduction bone harvester 100, as described herein.

Tubular attachment 52 includes an arm 68 extending therefrom. Arm 68 extends transverse to tubular attachment 52 to facilitate access to cavity 64, as shown in FIG. 2. An image guide, such as, for example, an emitter array 72 is disposed with arm 68. Emitter array 72 is configured for generating a signal to sensor array 74 of surgical navigation system 76 to provide a dynamic reference frame and position of surgical instrument 12 relative to tissue.

In some embodiments, the image guide may include human readable visual indicia, human readable tactile indicia, human readable audible indicia, one or more components having markers for identification under x-ray, fluoroscopy, CT or other imaging techniques, at least one light emitting diode, a wireless component, a wired component, a near field communication component and/or one or more components that generate acoustic signals, magnetic signals, electromagnetic signals and/or radiologic signals. In some embodiments, navigation component 50 is connected with tube 16 via an integral connection, friction fit, pressure fit, interlocking engagement, mating engagement, dovetail connection, clips, barbs, tongue in groove, threaded, magnetic, key/keyslot and/or drill chuck.

In some embodiments, the signal generated by emitter array 72 represents a position of surgical instrument 12 relative to tissue, such as, for example, bone. In some embodiments, the signal generated by emitter array 72 represents a three-dimensional position of surgical instrument 12 relative to tissue. In some embodiments, emitter array 72 may include a reflector array configured to reflect a signal from sensor array 74.

In some embodiments, sensor array 74 receives signals from emitter array 72 to provide a three-dimensional spatial position and/or a trajectory of surgical instrument 12 relative to tissue. Emitter array 72 communicates with a processor of a computer 78 of navigation system 76 to generate data for display of an image on a monitor 80, as described herein. In some embodiments, sensor array 74 receives signals from emitter array 72 to provide a visual representation of a position of surgical instrument 12 relative to tissue. See, for example, similar surgical navigation components and their use as described in U.S. Pat. Nos. 6,021,343, 6,725,080, 6,796,988, the entire contents of each of these references being incorporated by reference herein.

Surgical navigation system 76 is configured for acquiring and displaying medical imaging, such as, for example, x-ray images appropriate for a given surgical procedure. In some embodiments, pre-acquired images of a patient are collected. In some embodiments, surgical navigation system 76 can include an O-Arm® imaging device 82 sold by Medtronic Navigation, Inc. having a place of business in Louisville, Colo., USA. Imaging device 82 may have a generally annular gantry housing that encloses an image capturing portion 84.

In some embodiments, navigation system 76 comprises image capturing portion 84 that may include an x-ray source or emission portion and an x-ray receiving or image receiving portion located generally or as practically possible 180 degrees from each other and mounted on a rotor (not shown) relative to a track of image capturing portion 84. Surgical navigation system 76 can include those disclosed in U.S. Pat. Nos. 8,842,893, 7,188,998; 7,108,421; 7,106,825; 7,001,045; and 6,940,941; the entire contents of each of these references being incorporated by reference herein.

In some embodiments, surgical navigation system 76 can include C-arm fluoroscopic imaging systems, which can generate three-dimensional views of a patient. The position of image capturing portion 84 can be precisely known relative to any other portion of an imaging device of navigation system 76. In some embodiments, a precise knowledge of the position of image capturing portion 84 can be used in conjunction with a tracking system 86 to determine the position of image capturing portion 70 and the image data relative to the patient. In some embodiments, the EM tracking system can include the STEALTHSTATION® AXIEM™ Navigation System, sold by Medtronic Navigation, Inc. having a place of business in Louisville, Colo. Exemplary tracking systems are also disclosed in U.S. Pat. Nos. 8,057,407, 5,913,820, 5,592,939, the entire contents of each of these references being incorporated by reference herein.

Bone harvester 100 includes a shaft 102. Shaft 102 extends between an end 104 and an end 106, and defines an axis A3. Shaft 102 is translatable relative to tube 16 within channel 32. Axis A3 is disposed co-axial with axis A1 during insertion of shaft 102. End 104 includes a tissue gathering element 108, which has a surface 110 that defines a cavity 112. Element 108 includes a cutting surface 116 for harvesting bone and/or tissue. In some embodiments, element 108 includes a trephine 116 to remove a circle of tissue or bone for harvesting bone and/or tissue. In some embodiments, element 108 includes a cutting surface configuration, such as, for example, hole saw, serrated edge, blade and/or scoop. In some embodiments, shaft 102 includes graduations for measuring depth and/or volume of harvested tissue.

Tube 16 guides and/or directs shaft 102 to orient element 108 in alignment with the selected tissue site. Element 108 is configured to harvest bone and/or tissue by cutting the bone and/or tissue and storing in cavity 112. Harvester 100 is removed from tube 16 to allow the bone and/or tissue to be harvested for use in a surgical procedure, or biopsied for pathological analysis. The bone and/or tissue is extruded from cavity 112 for use in the surgical procedure, or biopsied for pathological analysis. End 106 includes one or a plurality of actuator handles 120. Handles 120 include a knurled surface to facilitate gripping by a practitioner. In some embodiments, a tamp 122 is utilized to extrude the bone and/or tissue obtained from cavity 112.

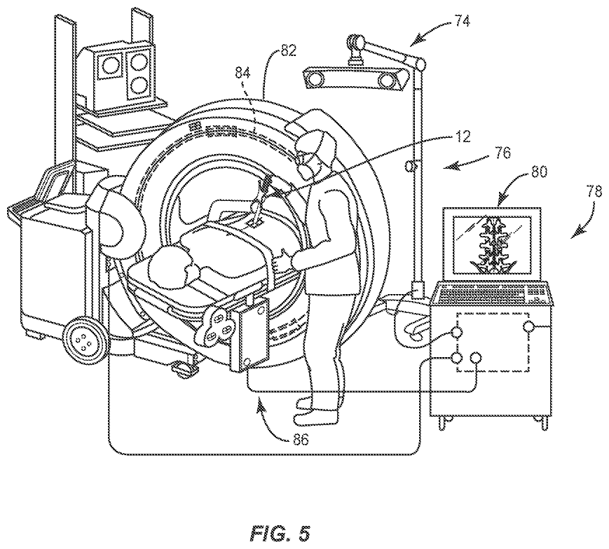

In one embodiment, as shown in FIGS. 5-9, surgical system 10, similar to the systems and methods described herein, is employed in connection with one or more surgical procedures, as described herein. The components of surgical system 10 can be employed with a surgery for treatment of an applicable condition or injury of an affected section of a spinal column and adjacent areas within a body, such as, for example, vertebrae V. To treat a selected section of vertebrae V, a medical practitioner obtains access to a surgical site including vertebrae V in any appropriate manner, such as through incision and retraction of tissues. In some embodiments, the components of surgical system 10 can be used in any existing surgical method or technique including open surgery, mini-open surgery, minimally invasive surgery and percutaneous surgical implantation, whereby vertebrae V are accessed through a mini-incision, or sleeve that provides a protected passageway to the area. Once access to the surgical site is obtained, the particular surgical procedure can be performed for treating the spine disorder.

Initially, surgical system 10 is employed with a method for treating a spine that includes a preparation and/or a pre-operative step of performing a three-dimensional scan of the patient anatomy, for example, the spine. In some embodiments, the preparation step may include verification of surgical instruments, draping and/or camera positioning. In some embodiments, the preparation step includes utilizing O-Arm® imaging device 82 in the operating room to obtain the three-dimensional images of the patient anatomy intra-operatively and/or prior to surgery, as shown in FIG. 5.

In some embodiments, a cannula is percutaneously passed through a layer of soft tissue, including skin into the posterior superior iliac spine (PSIS), accessed through a posterior portion of the patient. The cannula is positioned percutaneously by positioning a dilator through the cannula and simultaneously inserting both members through the soft tissue.

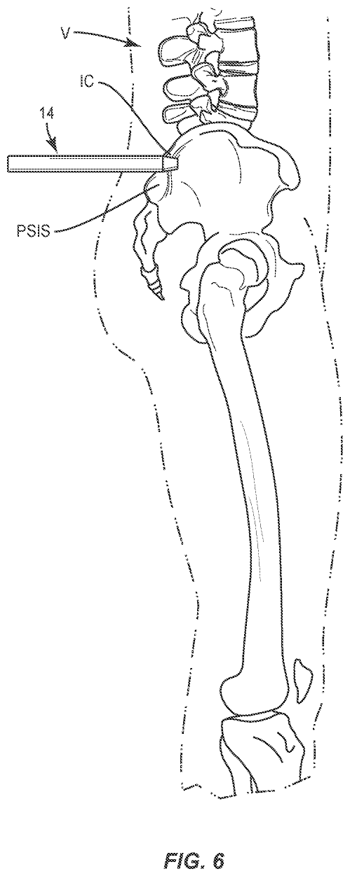

Once the cannula is positioned adjacent or relative to the PSIS, the dilator can be removed from the cannula. Once the dilator is removed from the cannula, the bore defined by the cannula can be used to position guide 14. Guide 14 is passed through the incision and through the layer of soft tissue, including skin and into engagement with the PSIS, as shown in FIG. 6. Guide 14 is percutaneously passed through the incision and through a layer of soft tissue, including skin and into engagement with the iliac crest IC. Pin 24 penetrates and engage tissue. Pin 24 secures guide 14 with the iliac. Pin 24 stabilizes guide 14 with the patient to resist and/or prevent relative movement.

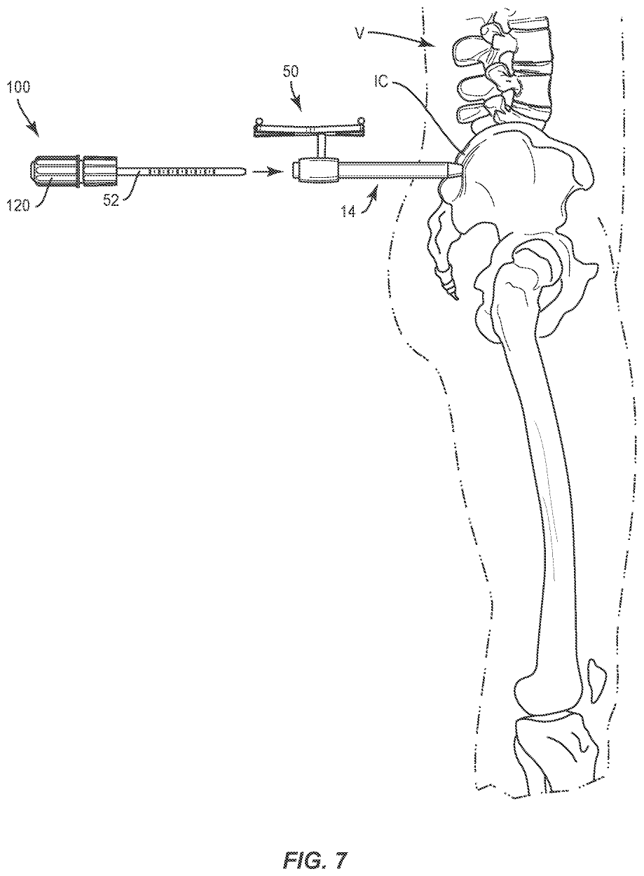

Tubular attachment 52 is assembled with tube 16, as described herein and shown in FIG. 7. Navigation component 50 communicates a signal to sensor array 74 to provide a dynamic reference frame and position of surgical instrument 12 relative to tissue. Navigation component 50 provides a signal representative of a position of tube 16 relative to a patient anatomy. Navigation component 50 includes a dynamic reference frame and provides location of the anatomy in a navigation field, as described herein.

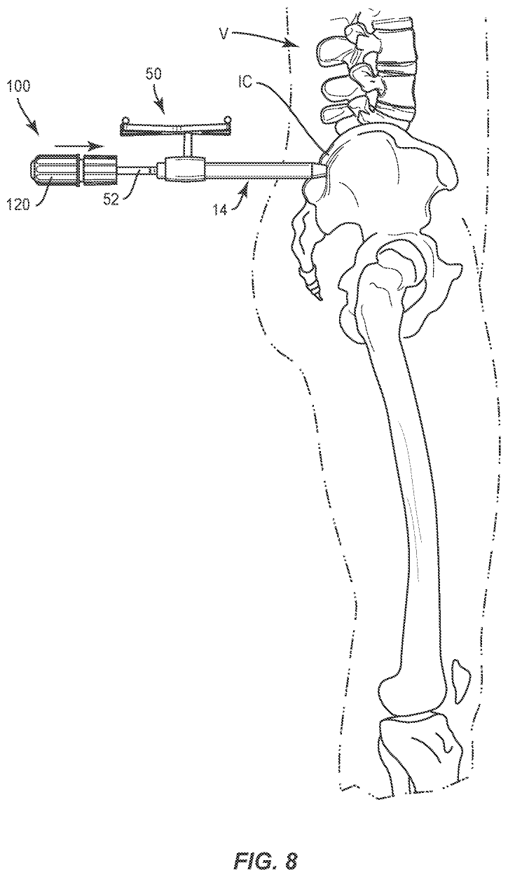

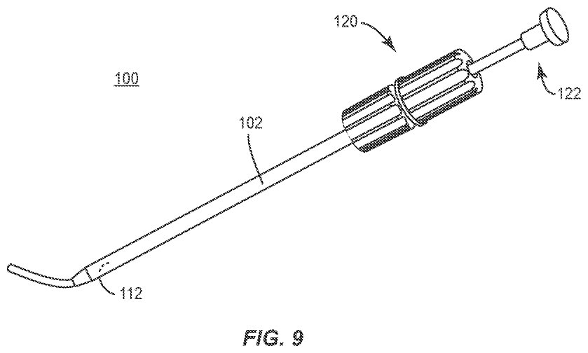

Harvester 100 is oriented for translation through tubular attachment 52 and tube 16, as shown in FIGS. 7 and 8. Harvester 100 is translated through channel 32 into engagement with tissue. Harvester 100 translates deeper into tissue than guide 14. Tube 16 guides and/or directs shaft 102 to orient element 108 in alignment with the selected tissue site. Handle 120 is manipulated causing element 108 to extract circular sections of tissue. The extracted tissue is stored in cavity 112. Harvester 100 is removed from tube 16. The bone and/or tissue is extruded from cavity 112, as shown in FIG. 9, for use in the surgical procedure, or biopsied for pathological analysis. Tamp 122 is utilized to extrude the bone and/or tissue obtained from cavity 112. Surgical instrument 12 allows for image-guided patient registration and dynamic tracking at the PSIS while providing for bone harvesting through a single incision. This allows the practitioner to harvest bone and/or tissue at any point during the procedure.

Upon completion of a procedure, the surgical instruments and non-implanted components of surgical system 10 are removed and the incision(s) are closed. One or more of the components of surgical system 10 can be made of radiolucent materials such as polymers. Radiomarkers may be included for identification under x-ray, fluoroscopy, CT or other imaging techniques. In some embodiments, the use of surgical navigation, microsurgical and image guided technologies may be employed to access, view and repair spinal deterioration or damage, with the aid of surgical system 10.

In some embodiments, surgical system 10 includes an agent, which may be disposed, packed, coated or layered within, on or about the components and/or surfaces of surgical system 10. In some embodiments, the agent may include bone growth promoting material, such as, for example, bone graft to enhance fixation of the fixation elements with vertebrae. In some embodiments, the agent may be HA coating. In some embodiments, the agent may include one or a plurality of therapeutic agents and/or pharmacological agents for release, including sustained release, to treat, for example, pain, inflammation and degeneration.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A surgical instrument comprising:
a tubular member including a tissue engaging element;
a shaft being disposable with and movable relative to the tubular member, the shaft including a tissue gathering element; and
an image guide connected with the tubular member and being oriented relative to a sensor for registration of anatomical image data and positional tracking of the anatomy during the procedure.

2. A surgical instrument as recited in claim 1, wherein the image guide includes a dynamic reference frame and the position includes location of the anatomy in a navigation field.

3. A surgical instrument as recited in claim 1, wherein the tissue engaging element includes a pin configured to penetrate tissue.

4. A surgical instrument as recited in claim 1, wherein the tissue engaging element includes a serrated edge disposed adjacent a distal end of the tubular member.

5. A surgical instrument as recited in claim 1, wherein the tubular member defines a channel and the shaft is translatable relative to the tubular member within the channel.

6. A surgical instrument as recited in claim 1, wherein the tissue engaging element is aligned with a selected tissue site of the anatomy and the tubular member includes an inner guide surface to orient the shaft in alignment with the selected tissue site.

7. A surgical instrument as recited in claim 6, wherein the tissue gathering element is removable from the tubular member to harvest tissue from the selected tissue site.

8. A surgical instrument as recited in claim 1, wherein the tissue gathering element includes a trephine.

9. A surgical instrument as recited in claim 1, wherein the tubular member and the shaft are disposed in a relative coaxial orientation.

10. A surgical instrument as recited in claim 1, wherein a proximal end of the shaft includes a knurled handle.

11. A surgical instrument as recited in claim 1, wherein the image guide includes a tubular attachment that defines a cavity configured for disposal of the tubular member.

12. A surgical instrument as recited in claim 11, wherein the tubular member is disposed in a fixed position with the attachment.

13. A surgical instrument as recited in claim 1, wherein the image guide comprises an emitter disposed in a fixed position with the tubular member.

14. A surgical instrument as recited in claim 1, wherein the image guide is oriented relative to a sensor to communicate a signal representative of a location of the anatomy in a navigation field and the sensor communicates with a processor to generate data for display of an image from a monitor.

15. A surgical instrument comprising:
an outer member including a pin configured to penetrate a selected tissue site of an anatomy;
a removable inner member being translatable relative to the outer member and including a tissue gathering element aligned with the selected tissue site; and
an image guide connected with the outer member and being oriented relative to a sensor for registration of anatomical image data and positional tracking of the anatomy during the procedure.

16. A surgical instrument as recited in claim 15, wherein the image guide includes a dynamic reference frame and the position includes location of the anatomy in a navigation field.

17. A surgical instrument as recited in claim 15, wherein the outer member includes an inner guide surface to orient the inner member in alignment with the selected tissue site.

18. A surgical instrument as recited in claim 15, wherein the outer member is disposed in a fixed position with the image guide.

19. A surgical system comprising:
a surgical instrument including an outer member having a tissue engaging element configured to penetrate a selected tissue site of an anatomy and an inner member being relatively translatable relative to the outer member, the inner member including a tissue gathering element aligned with the selected tissue site;
an image guide connected with the outer member and being oriented for registration of anatomical image data and positional tracking of the anatomy during the procedure in a navigation field; and
a tracking device including a sensor that receives the signal and communicates with a processor to generate data for display of an image from a monitor.

20. A surgical system as recited in claim 19, wherein the outer member is disposed in a fixed position with the image guide.
